(19) United States
(12) Patent Application Publication
(10) Pub. No.: US 2015/0299424 A1
(43) Pub. Date: Oct. 22, 2015

(54) FURFURYL-BASED ESTERS

(71) Applicant: POLYONE CORPORATION, Avon Lake, OH (US)

(72) Inventors: Roger W. AVAKIAN, Solon, OH (US); Yannan DUAN, Westlake, OH (US)

(73) Assignee: POLYONE CORPORATION, Avon Lake, OH (US)

(21) Appl. No.: 14/652,054

(22) PCT Filed: Dec. 12, 2013
(86) PCT No.: PCT/US13/74671

§ 371 Date: Jun. 12, 2015

Related U.S. Application Data
(60) Provisional application No. 61/737,012, filed on Dec. 13, 2012.

(51) Int. Cl. C08K 5/1535; C07D 307/42

(52) U.S. Cl. CPC C08K 5/1535; C07D 307/42

(57) ABSTRACT

The reactions are disclosed of furfuryl alcohol in the presence of catalyst with aliphatic diacid esters or aliphatic diacid anhydrides to synthesize diesters and monoesters, respectively. The reaction is disclosed of the monoester in the presence of catalyst with alkanols to synthesize diesters. The two types of diesters and the monoesters are proven to be suitable as plasticizers for polymers, particularly polylactic acid. Thermoplastic compounds are disclosed using the esters as plasticizers.

### CLAIM OF PRIORITY

This application claims priority from U.S. Provisional Patent Application Ser. No. 61/737,012 bearing Attorney Docket Number 12012024 and filed on Dec. 13, 2012, which is incorporated by reference.

### FIELD OF THE INVENTION

This invention concerns monoesters or diesters of furfuryl alcohol which can plasticize polymers, such as polylactic acid.

### BACKGROUND OF THE INVENTION

Plasticizers are a class of polymer modifiers that are very useful in many polymers to tailor their properties for specific applications, typically used to impart flexibility, as in polyvinyl alcohol (PVC) which is the largest single polymer that uses plasticizers. Plasticizers are also used in cellulosics, nylons, and in some polyesters. But by far PVC consumes the largest share of plasticizers with over 30 classes of chemical compounds used. It is PVC's ability to be effectively modified from a rigid transparent polymer to a flexible clear polymer and even to a polymer/plasticizer “solution” (plastisol) that makes PVC such a high volume, popular polymer worldwide.

However, it is becoming more desired to have plasticizers that are bio-derived to make their manufacture more sustainable. In addition some classes of plasticizers used in PVC such as phthalates have come under increased toxicological investigation and replacements are sought. Lastly cost effective raw materials are desired to make the transition to these more “green” plasticizers more commercially feasible.

At the same time, biopolymers are gaining traction as polymers for packaging, electronics, fibers, and films because of their lower carbon footprint versus traditional petro-chemically derived polymers. One such polymer is polylactic acid (PLA), which can be used in many applications where PVC was once preferred for use. PLA's high modulus allows its use as a rigid polymer in most of the applications named above, but it would be desired to have a plasticizer for PLA that is bio-derived, yields a flexible composition, retains clarity, and does not exude out over time. This would allow PLA to enter into applications now reserved for flexible PVC. As added benefits, it is desired that such a candidate plasticizer be easy to synthesize, be cost effective, and not cause any negative effects over time.

### SUMMARY OF THE INVENTION

Plasticizers act by having sufficient molecular interaction with the base polymer to allow polymer chains to slide by one another and effectively reducing the glass transition temperature (the temperature in which an amorphous polymer passes from a glassy region to a more rubbery region). However the interaction cannot be so strong as to be an actual solvent for the polymer. Also this interaction should be amendable at the high temperature of polymer blending with other components and not become less compatible at room temperature in which case it would “exude” out or be incompatible over time with the base polymer. Thus a balance of compatibility is desired.

What the art needs is an inexpensive, efficient plasticizer for PLA.

The present invention provides monoester and diester compositions of matter which are effective as plasticizers for PLA.

One aspect of the present invention is Formula I as follows:

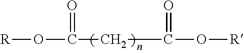

wherein n=2 to 22, inclusive; R=furfuryl; and R′=hydrogen, furfuryl, or an alkyl having from 1 to 10 carbon atoms. If R′ is hydrogen, then the chemical is a monoester. If R′ is either furfuryl or an alkyl having from 1 to 10 carbon atoms, then the chemical is a diester.

Preferably, the composition of matter selected from the group consisting of 1,4-bis(furan-2-ylmethyl) butanedioate, 4-(furan-2-ylmethoxy)-4-oxobutanoic acid, and 1-butyl 4-furan-2-ylmethyl butanedioate.

Another aspect of the present invention is the new composition of matter called 1,4-bis(furan-2-ylmethyl) butanedioate.

Another aspect of the present invention is the new composition of matter called 4-(furan-2-ylmethoxy)-4-oxobutanoic acid.

Another aspect of the present invention is the new composition of matter called 1-butyl 4-furan-2-ylmethyl butanedioate.

Another aspect of the present invention is a method of synthesizing 1,4-bis(furan-2-ylmethyl) butanedioate by an organometallic-catalyzed transesterification reaction of furfuryl alcohol and diethyl succinate in the presence of titanium isopropoxide.

Another aspect of the present invention is a method of synthesizing 4-(furan-2-ylmethoxy)-4-oxobutanoic acid by base-catalyzed ring-opening esterification reaction of furfuryl alcohol and succinic anhydride in the presence of a catalyst selected from the group consisting of pyridine, triethylamine, and dimethylaminopyridine.

Another aspect of the present invention is a method of synthesizing 1-butyl 4-furan-2-ylmethyl butanedioate by acid-catalyzed direct esterification reaction of a furfuryl monoester and 1-butanol in the presence of p-toluene sulfonic acid.

Another aspect of the present invention is uses of the compositions of matter identified above in mixture with a polymer, preferably polylactic acid or as reactants themselves with other chemicals.

Embodiments of the invention are explained with reference to the drawings.

### BRIEF DESCRIPTION OF THE DRAWING

FIG. 1 is a schematic of the reaction equipment employed to prepare the new compositions of matter identified as Examples 1 and 5.

FIG. 2 is a schematic of the reaction equipment employed to prepare the new compositions of matter identified as Examples 2-4.

FIG. 3 is the organometallic-catalyzed transesterification reaction equation for Example 1 to prepare 1,4-bis(furan-2-ylmethyl) butanedioate, one embodiment of a diester of the invention.

FIG. 4 is the base-catalyzed ring-opening esterification reaction equation for Examples 2-4 to prepare 4-(furan-2-ylmethoxy)-4-oxobutanoic acid, one embodiment of a monoester of the invention.

FIG. 5 is the acid-catalyzed direct esterification reaction equation for Example 5 to prepare 1-butyl 4-furan-2-ylmethyl butanedioate, one embodiment of another diester of the invention.

FIG. 6 is a chart of weight gain over time of the compositions of Examples 1-5 in PLA, one test for use of such compositions as plasticizers for polymers, such as PLA.

### EMBODIMENTS OF THE INVENTION

Furfuryl Alcohol

Furfuryl alcohol is one of the direct or indirect starting ingredients for the various embodiments of the invention. The IUPAC name is 2-furanmethanol. Its CAS No. is 98-0-0. It is C₅H₆O₂ and has a molecular weight of 98.10. It has a melting point of −31° C., a specific gravity of 1.129, and a boiling point of 171° C. It is miscible in water. It can be obtained from bio-derived sources, including corn cobs and sugar cane bagasse.

Commercially, furfuryl alcohol is available from Penn A Kem of Memphis, Tenn., USA.

Diester Compositions from Transesterification of Furfuryl Alcohol and Aliphatic Diacid Esters

New diester compositions of this invention can be prepared generally by the transesterification reaction of furfuryl alcohol in the presence of an organometallic catalyst with a reagent comprising an aliphatic diacid ester having from 4 to 12 carbon atoms. The molar ratio of the reaction of furfuryl alcohol and aliphatic acid ester is 2:1.

Ester derivatives of the aliphatic diacids include oxalates; malonates, such as diethyl malonate; succinates, such as diethyl succinate (C₈H₁₄O₄ and CAS No. 123-25-1); glutarates; adipates; pimelates; and suberates. Diethyl succinate is currently preferred.

Any organometallic catalyst is a candidate for use as the catalyst for this transesterification reaction synthesis of the diester. Nonlimiting examples are titanium isopropoxide, zirconium isopropoxide, Fascat® 4101 tin-based catalyst, with titanium isopropoxide being preferred. The amount of catalyst can range from about 0.05 to about 3.0 and preferably from about 0.5 to about 1.0 weight percent to the combined weight of the resulting diester composition including the catalyst.

Commercially, diethyl succinate is available from Vertellus of Indianapolis, Ind. and Sigma Aldrich.

Commercially, titanium isopropoxide is available from Dorf Ketal of Houston, Tex., USA. The Fascat® tin catalyst is available from Arkema.

Monoester Compositions from Ring-Opening Esterification of Furfuryl Alcohol and Aliphatic Diacid Anhydrides

New monoester compositions of this invention can be prepared generally by the ring-opening esterification reaction of furfuryl alcohol in the presence of a base catalyst with a reagent comprising an aliphatic diacid anhydride having from 4 to 8 carbon atoms. The molar ratio of furfuryl alcohol and aliphatic diacid anhydride is 1:1.

A non-limiting examples of aliphatic diacid anhydrides includes succinic diacid anhydride (IUPAC named as oxolane-2,5-dione; CAS No. 108-30-5) commercially available from Dixie Chemical Company of Houston, Tex., USA; glutaric diacid anhydride, adipic diacid anhydride, pimelic diacid anhydride, suberic diacid anhydride. Preferred are those aliphatic diacid anhydrides which are bio-derived.

Any base catalyst is a candidate to be the catalyst for this monoester synthesis. Non-limited examples can be selected from the group consisting of pyridine, dimethylaminopyridine, and triethylamine. The amount of monoester catalyst can range from about 1 to about 20 and preferably from about 3 to about 10 weight percent to the combined weight of the resulting monoester composition including the catalyst.

Commercially, pyridine and dimethylaminopyridine are available from Vertellus Chemicals, and triethylamine is available from Dow Chemical or ICC Chemical.

Diester Compositions from Direct Esterification of Monoester Compositions and Alkanols

The new monoester compositions described above or other aliphatic monoesters of similar structure can be reacted via direct esterification in the presence of an acid catalyst with an aliphatic alcohol having from 1 to 10 carbon atoms to make an alternative embodiment of the new diester compositions.

Non-limiting examples of the aliphatic alcohols include methanol, ethanol, 1-propanol, isopropanol, 1-butanol, pentanol of the various forms, and hexanol of the various forms, with 1-butanol being preferred and commercially available from Sigma-Aldrich, among others. Bio-derived alkanols are known, including bio-derived 1-butanol.

Any acid catalyst is a candidate to be the catalyst for this diester synthesis. Non-limited examples can be selected from the group consisting of p-toluene sulfonic acid (PTSA), methane sulfonic acid, sulfuric acid, montmorillonite (acid form), acidic ion exchange resin, acidic fluorinated resin, with PTSA being preferred and commercially available from Evonik A.G. The amount of diester catalyst can range from about 0.1 to about 3.0 and preferably from about 0.5 to about 1.0 weight percent to the combined weight of the resulting diester composition including the catalyst.

Optional Quenching

Optionally, but preferably, it has been found that quenching of the catalyst in the first type of reaction identified above can assist in the stabilization of the new compositions after their synthesis. Additionally, the use of quenching agents can reduce the incidence of coloration other than a clear plasticizer which is preferred for cosmetic reasons in a thermoplastic polymer.

It has been found in the Examples below for the organometallic-catalyzed transesterification reaction that quenching dissociates the titanium catalyst from the furfuryl alcohol, thereby removing potential unacceptable discoloration of the resulting diester composition. Not all coloration is removed, but an acceptable plasticizer candidate has been achieved.

Non-limiting examples of quenching agents include phenylphosphinic acid and phenylphosphonic acid.

The amount of quenching agent to be added to the transesterification reaction should be that amount necessary to achieve a molar ratio of quenching agent to titanium catalyst equaling about three to one (3:1) and preferably as close to 3.0:1.0 as possible.

No quenching agent is needed for the ring-opening esterification reaction because the base catalyst can be washed from the resulting monoester composition. Also, no quenching agent is needed for the direct esterification reaction because the acid catalyst can be washed away.

Quenching agent can be added to the plasticizer before melt-mixing or during melt-mixing with the thermoplastic polymer either in batch or continuous operation.

Examples below explain the synthesis of three new furfuryl-based esters, both providing details of such syntheses and also providing reaction principles from which a person having ordinary skill in the art without undue experimentation can identify other species of furfuryl-based esters using the categories of reagents described above.

These monoester or diester compositions can then be characterized for their plasticization effects upon polymers, such as polylactic acid, a bio-derived polymer resin which truly needs a low expensive, effective plasticizer also of bio-derived origins. As such, the novel compositions can be plasticizers in thermoplastic compounds.

Plasticized Polymer Compounds

Polylactic Acid

PLA is a well-known biopolymer, having the following monomeric repeating group in the following formula:

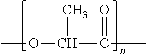

The PLA can be either poly-D-lactide, poly-L-lactide, or a combination of both. The amount of each enantiomer can affect solubility parameters, as explained below.

PLA is commercially available from NatureWorks, LLC located in all manufacturing regions of the world. Any grade of PLA is a candidate for use in the present invention. Currently, grades 4042D, 4032D, and 4060D are preferred. The number average molecular weight of PLA can be any which is currently available in a commercial grade or one which is brought to market in the future. To the extent that a current end use of a plastic article could benefit from being made from PLA, then that suitable PLA should be the starting point for constructing the compound of the present invention.

Polyvinyl Chloride

Polyvinyl chloride polymers are widely available throughout the world. Polyvinyl chloride resin as referred to in this specification includes polyvinyl chloride homopolymers, vinyl chloride copolymers, graft copolymers, and vinyl chloride polymers polymerized in the presence of any other polymer such as a heat distortion temperature (HDT) enhancing polymer, impact toughener, barrier polymer, chain transfer agent, stabilizer, plasticizer, or flow modifier.

In the practice of the invention, there may be used polyvinyl chloride homopolymers or copolymers of polyvinyl chloride comprising one or more comonomers copolymerizable therewith. Suitable comonomers for vinyl chloride include acrylic and methacrylic acids; esters of acrylic and methacrylic acid, wherein the ester portion has from 1 to 12 carbon atoms, for example methyl, ethyl, butyl and ethylhexyl acrylates and the like; methyl, ethyl and butyl methacrylates and the like; hydroxyalkyl esters of acrylic and methacrylic acid, for example hydroxymethyl acrylate, hydroxyethyl acrylate, hydroxyethyl methacrylate and the like; glycidyl esters of acrylic and methacrylic acid, for example glycidyl acrylate, glycidyl methacrylate and the like; alpha, beta unsaturated dicarboxylic acids and their anhydrides, for example maleic acid, fumaric acid, itaconic acid and acid anhydrides of these, and the like; acrylamide and methacrylamide; acrylonitrile and methacrylonitrile; maleimides, for example, N-cyclohexyl maleimide; olefin, for example ethylene, propylene, isobutylene, hexene, and the like; vinylidene chloride, for example, vinylidene chloride; vinyl ester, for example vinyl acetate; vinyl ether, for example methyl vinyl ether, allyl glycidyl ether, n-butyl vinyl ether and the like; crosslinking monomers, for example diallyl phthalate, ethylene glycol dimethacrylate, methylene bis-acrylamide, tracrylyl triazine, divinyl ether, allyl silanes and the like; and including mixtures of any of the above comonomers.

The present invention can also use chlorinated polyvinyl chloride (CPVC), wherein PVC containing approximately 57% chlorine is further reacted with chlorine radicals produced from chlorine gas dispersed in water and irradiated to generate chlorine radicals dissolved in water to produce CPVC, a polymer with a higher glass transition temperature (Tg) and heat distortion temperature. Commercial CPVC typically contains by weight from about 58% to about 70% and preferably from about 63% to about 68% chlorine. CPVC copolymers can be obtained by chlorinating such PVC copolymers using conventional methods such as that described in U.S. Pat. No. 2,996,489, which is incorporated herein by reference. Commercial sources of CPVC include Lubrizol Corporation.

The preferred resin is a polyvinyl chloride homopolymer. Commercially available sources of polyvinyl chloride polymers include OxyVinyls LP of Dallas, Tex. and Shintech USA of Freeport, Tex.

The amount of plasticizer of the invention in the polymer can range from about 10 parts to about 100 parts per hundred of polymer resin and preferably from about 15 parts to about 35 parts per hundred of polymer resin. Stated alternatively, the amount of plasticizer of the invention in the polymer compound can range from about 9 weight percent to about 50 weight percent of the compound and preferably from about 13 weight percent to about 26 weight percent of the compound.

Optional Additives for Plasticized Thermoplastic Compounds

The compound of the present invention can include conventional plastics additives in an amount that is sufficient to obtain a desired processing or performance property for the compound. The amount should not be wasteful of the additive or detrimental to the processing or performance of the compound. Those skilled in the art of thermoplastics compounding, without undue experimentation but with reference to such treatises as Plastics Additives Database (2004) from Plastics Design Library (www.elsevier.com), can select from many different types of additives for inclusion into the compounds of the present invention.

Non-limiting examples of optional additives include adhesion promoters; biocides (antibacterials, fungicides, and mildewcides), anti-fogging agents; anti-static agents; bonding, blowing and foaming agents; crosslinking agents; dispersants; fillers and extenders; fire and flame retardants and smoke suppresants; impact modifiers; initiators; lubricants; micas; pigments, colorants and dyes; plasticizers; processing aids; release agents; silanes, titanates and zirconates; slip and anti-blocking agents; stabilizers; stearates; ultraviolet light absorbers; viscosity regulators; waxes; and combinations of them.

Processing of Thermoplastic Compounds

The preparation of compounds of the present invention is uncomplicated. The compound of the present can be made in batch or continuous operations.

Mixing in a continuous process typically occurs in an extruder that is elevated to a temperature that is sufficient to melt the polymer matrix with addition either at the head of the extruder or downstream in the extruder of the solid ingredient additives. Extruder speeds can range from about 50 to about 500 revolutions per minute (rpm), and preferably from about 100 to about 300 rpm.

Introduction of plasticizer into the batch or continuous process can be later to a batch melt-mixing apparatus or at a downstream liquid injection port in a continuous melt-mixing apparatus. Later and downstream addition is conventional to reduce heated residence time of the plasticizer in the hot apparatus and to delay introduction until after the solid polymer resin material has been melted for better dispersion of plasticizer in resin. Typically, the output from the extruder is pelletized for later extrusion or molding into polymeric articles.

Mixing in a batch process typically occurs in a Banbury mixer that is also elevated to a temperature that is sufficient to melt the polymer matrix to permit addition of the solid ingredient additives. The mixing speeds range from 60 to 1000 rpm and temperature of mixing can be ambient. Also, the output from the mixer is chopped into smaller sizes for later extrusion or molding into polymeric articles.

Subsequent extrusion or molding techniques are well known to those skilled in the art of thermoplastics polymer engineering. Without undue experimentation but with such references as “Extrusion, The Definitive Processing Guide and Handbook”; “Handbook of Molded Part Shrinkage and Warpage”; “Specialized Molding Techniques”; “Rotational Molding Technology”; and “Handbook of Mold, Tool and Die Repair Welding”, all published by Plastics Design Library (www.elsevier.com), one can make articles of any conceivable shape and appearance using compounds of the present invention.

### Usefulness of the Invention

Plasticizer Uses

Use of plasticizers in thermoplastic compounds increases flexibility (flexural modulus), stretch, (tensile strength and elongation), softness (durometer hardness), among other properties. Rigid thermoplastic resins made more flexible render such resins useful in a wide variety of industries, such as the wire and cable industry for insulation and jacketing, the automotive industry for instrument panels and other interior components, the consumer industry for shower curtains, the appliance industry for flexible parts, and other industries.

The plasticized thermoplastic compounds can be formed a plastic article of any shape, using such formation techniques as extrusion, molding, calendering, thermoforming, casting, dipping, powder coating, additive manufacturing via fused deposition modeling, and other polymer shaping techniques.

The plasticizer can partially solubilize the polymer resin, described in relation to solubility parameters, used in the Examples below.

At its most basic, the plasticizer loosens the entangled polymer macromolecules, yet retaining a solid structure. At one extreme, the plasticizer and polymer form a solid solution, called a plastisol.

The polymer processing art is quite familiar with vinyl plastisols. These plastisols are formed from dispersion-, microsuspension-, and emulsion-grade poly(vinyl chloride) (PVC) resins (homopolymers and copolymers) and plasticizers. Exemplary dispersion-grade PVC resins are disclosed in U.S. Pat. Nos. 4,581,413; 4,693,800; 4,939,212; and 5,290,890, among many others such as those referenced in the above four patents.

The monoester and diester compositions of this invention are particularly suitable as plasticizers for bio-derived resins such as polylactic acid (PLA), because the starting ingredients for the monoesters and diesters are themselves available from bio-derived sources. Thus, for those desiring sustainable thermoplastic compounds, one can use one of the compositions as a plasticizer for PLA, making a totally bio-derived combination.

Monomer Uses

The composition bearing the formula below can also serve as a polymeric building block, depending on what and R′ is and depending on reagents are brought into contact with the composition.

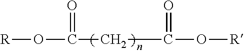

wherein n=2 to 22, inclusive; R=furfuryl; and R′=hydrogen, furfuryl, or an alkyl having from 1 to 10 carbon atoms.

The furfuryl endgroup R can react with any functionalized second chemical including functionalized polymers themselves. The furfuryl moiety can also react with maleimides or bismaleimides, even in a reversible manner, which can be used in a variety of environments having two different, controllable conditions.

The endgroup R′ can also react with a variety of chemicals having functional groups including functional polymers themselves. If R′ is furfuryl, the chemical can be a maleimide, a bismaleimide, or other dienophile. If R′ is hydrogen, the chemical can have a functional group selected from the group consisting of an alcohol, an oxirane, an amine, a metallic ion, a hydroxide, a silane and a silanol. If R′ is an alkyl, the chemical can be an ester reactive via transesterification in the presence of a catalyst.

### EXAMPLES

### Evaluation Equipment and Software

Gas chromatographymass spectrometry (GC-MS) was utilized to analyze the structure of intermediate using HP 6890 series GC system and HP 5943 mass-selective detector.

Büchi® rotavapor R-215 digital rotary evaporator was utilized to remove solvents of a solution as a purification method.

Molecular Modeling Pro 6.33 (ChemSW, Inc.) was utilized to calculate the theoretical Hansen's 3D solubility parameters of each plasticizer, to suggest which structure is favored as a plasticizer.

### Example 1

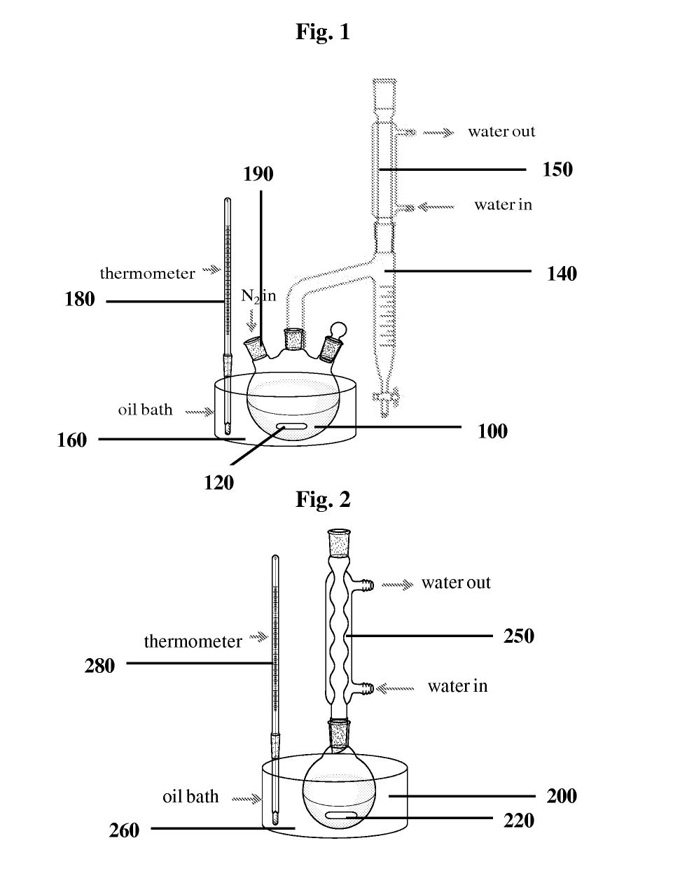

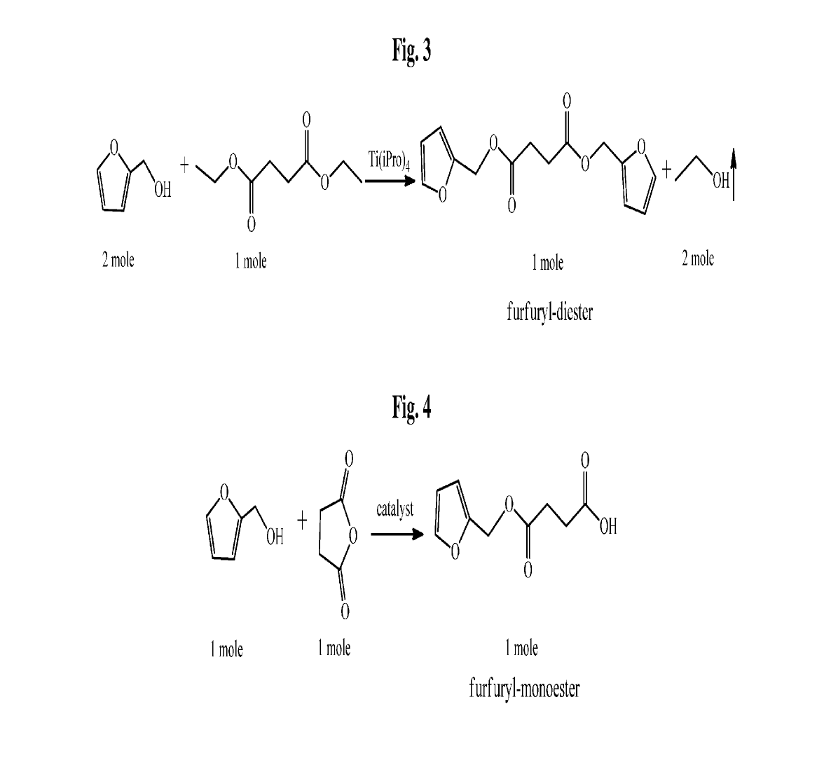

A furfuryl-based diester was synthesized via transesterification reaction using furfuryl alcohol and diethyl succinate in the presence of a catalyst. First, 20.11 grams of furfuryl alcohol (0.205 mole) was added into a 250 mL three-neck round bottom flask 100 along with a magnetic stirring bar 120, as seen in FIG. 1. Then 17.50 grams of diethyl succinate (0.100 mole) was added and mixed with furfuryl alcohol by shaking the flask. The mixture was a light yellow colored viscous liquid. The flask was equipped with a Dean-Stark apparatus 140 connected with a Liebig water-cooled condenser 150. Then the flask 100 was heated in an oil bath 160 to 80° C. measured using thermometer 180. Then 0.18 grams of titanium isopropoxide (Ti(iPro)₄, 0.5 wt %) was added into the flask. The mixture turned amber color once after the catalyst was added. The reaction proceeded under dry nitrogen flow into opening 190 to help removal of ethanol for 2-3 hours. The reaction continued until the clear liquid collected in Dean-Stark apparatus 140 no longer accumulated. The clear liquid was weighed to be 9.0 grams. The final product was a viscous liquid in amber color. The transesterification reaction scheme is shown in FIG. 3.

A sample from Example 1 was diluted with tetrahydrofuran and analyzed by gas chromatography—mass spectrometry [GC/MS]. The analysis conditions are provided in Table 1.

TABLE 1

| Parameter | Conditions |
|---|---|
| Instrument | Agilent 6890/5973 MSD |
| Column | 30 meter × 0.25 mm RTX-5 with 0.25 micron film |
| | thickness |
| Injection | 2 μL - split ratio 25:1 |
| Carrier | Helium - constant flow @ 1.2 mL/min |
| Program | 50° C. hold 5 minutes, 20° C./minute ramp to 310° C.; hold |
| | 5 minutes |
| Detector | MSD; transfer line 280° C.; source 230° C.; Quad 150° C. |

Peak identification was made based on either a match from the mass spectrum of the peak with commercial database collections (Wiley/NIST) or from similarity with commercial spectra and other data obtained from the analysis. The resulting chromatogram showed 5 main peaks. They are listed in Table 2 in order of their relative peak heights from the total ion chromatogram.

TABLE 2

| Time | | |
|---|---|---|
| (minutes) | Identification | Comments |
| 14.9 | Diester of succinic acid and | Ions = 53, 81, 97, 98, 101, |
| | furfuryl alcohol | 160, 278 |
| 12.8 | Mixed ester of succinic acid and | Ion = 53, 81, 97, 101, 129, |
| | furfuryl alcohol + ethanol | 226 |
| 12.5 | Butylated hydroxyl toluene | Library Match |
| | (from solvent) | |
| 5.6 | Furfuryl alcohol | Library match |
| 10.0 | Diethyl succinate | Library match |

The spectrum of the peak at 14.9 showed fragments at 53, 81 and 97 which are from furfuryl alcohol and the 101 is characteristic of succinate esters. A very weak molecular ion was detected at 278, which was consistent with the desired furfuryl diester product.

The peak at 12.8 had a mass spectrum with ions expected for a furfuryl ester of succinic acid. The additional ions (129) are consistent with an ethyl ester. This combination, along with the molecular weight of 226, is consistent with a mixed furfuryl/ethyl ester.

The IUPAC name for the product of Example 1 is 1,4-bis(furan-2-ylmethyl) butanedioate.

### Example 2

A furfuryl-based monoester was synthesized via esterification reaction by reacting furfuryl alcohol and succinic anhydride in the presence of a catalyst. First, 4.91 grams of furfuryl alcohol (0.050 mole) was dissolved in 30 mL of acetone in a 100 mL single-neck round bottom flask 200 along with a magnetic stirring bar 220, as seen in FIG. 2. Then 5.05 grams of succinic anhydride (0.050 mole) was added into the flask, and it took about 10 minutes for the white solid to dissolve in the acetone solution. The mixture was a yellow-colored solution. The flask was equipped with a Liebig water-cooled condenser 250. Then 0.40 grams of pyridine (0.005 mole) was added dropwise into the solution. The flask was heated in an oil bath 260 to 65° C. measured using thermometer 280. The solution was refluxed for 5 hours before the reaction was stopped. The solution was clear and in an amber color. The solvent was removed by a rotary evaporator (Buchi rotavapor R-215, not shown). The final product was an amber colored viscous liquid. The pH value of the final product was pH ˜7, tested by pH test paper. The esterification reaction scheme is shown in FIG. 4.

A sample of Example 2 was diluted with acetonitrile and analyzed by gas chromatography—mass spectrometry [GC/MS]. The analysis conditions are in Table 3.

TABLE 3

| Parameter | Conditions |
|---|---|
| Instrument | Agilent 6890/5973 MSD |
| Column | 30 meter × 0.25 mm RTX-5 with 0.25 micron film |
| | thickness |
| Injection | 2 μL - split ratio 25:1 |
| Carrier | Helium - constant flow @ 1.2 mL/min |
| Program | 40° C. hold 5 minutes, 20° C./minute ramp to 310° C.; |
| | hold 5 minutes |
| Detector | MSD; transfer line 280° C.; source 230° C.; Quad 150° C. |

Peak identification was made based either on a match of the mass spectrum of the peak with that found in a commercial database collection (Wiley/NIST) or from similarity with commercial spectra and other data obtained from the analysis. The resulting chromatogram showed 4 main peaks. They are listed in Table 4 in order of their relative peak heights from the total ion chromatogram. Peak heights are not necessarily an indication of relative concentration.

TABLE 4

| Time | | |
|---|---|---|
| (minutes) | Identification | Comments |
| 13.1 | Monoester of succinic anhydride and | Ion = 53, 81, 97, 98, |
| | furfuryl alcohol | 101, 198 |
| 15.4 | Diester of succinic anhydride and | Ions = 53, 81, 97, |
| | furfuryl alcohol | 98, 101, 160 |
| 9.5 | Ethyl levulinate | Library Match |
| 4.0 | Pyridine | Library match |

The peak at 13.1 minutes showed a molecular ion consistent with the expected molecular weight of the monoester (198). In addition, the peak was slightly tailed, characteristic of acids. The ions detected were also consistent with furfuryl alcohol (53, 81, 97, 98) and succinic acid (100, 101).

The peak at 15.4 minutes showed no molecular ion. The ions from the mass spectrum were similar to that seen for the peak at 13.1 minutes (monoester). The elution time was longer than the monoester, and the peak shape was more Gaussian. There were no other components in the mixture that would react with an acid other than the furfuryl alcohol. These observations are consistent with the indicated diester.

The IUPAC name for the monoester product of Example 2 is 4-(furan-2-ylmethoxy)-4-oxobutanoic acid.

### Example 3

A furfuryl-based monoester was synthesized via esterification reaction by reacting furfuryl alcohol and succinic anhydride in the presence of a catalyst. First, 4.91 grams of furfuryl alcohol (0.050 mole) was dissolved in 30 mL of acetone in a 100 mL single-neck round bottom flask 200 along with a magnetic stirring bar 220, as seen in FIG. 2. Then 5.15 grams of succinic anhydride (0.051 mole) was added into the flask 200, and it took about 10 minutes for the white solid to dissolve in the acetone solution. The mixture was a yellow-colored solution. The flask was equipped with a Liebig water-cooled condenser 250. Then 0.51 grams of triethylamine (TEA, 0.005 mole) was added dropwise into the solution. The flask was heated in an oil bath 260 to 65° C. as measured using thermometer 280. The solution was refluxed for 5 hours before the reaction was stopped. The solution was clear and in an amber color. The solvent was removed by a rotary evaporator (Buchi rotavapor R-215, not shown). The final product was an amber colored viscous liquid. The pH value of the final product was pH ˜7, tested by pH test paper. The esterification reaction scheme is shown in FIG. 4.

A sample of Example 3 was diluted with tetrahydrofuran and analyzed by gas chromatography—mass spectrometry [GC/MS]. The analysis conditions are in Table 5.

TABLE 5

| Parameter | Conditions |
|---|---|
| Instrument | Agilent 6890/5973 MSD |
| Column | 30 meter × 0.25 mm RTX-5 with 0.25 micron film thickness |
| Injection | 2 μL - split ratio 25:1 |
| Carrier | Helium - constant flow @ 1.2 mL/min |
| Program | 50° C. hold 5 minutes, 10° C./minute ramp to 300° C.; hold 5 |
| | minutes |
| Detector | MSD; transfer line 280° C.; source 230° C.; Quad 150° C. |

Peak identification was made based on either a match from the mass spectrum of the peak with commercial database collections (Wiley/NIST) or from similarity with commercial spectra and other data obtained from the analysis. The resulting chromatogram showed 4 main peaks. They are listed in Table 6 in order of their relative peak heights from the total ion chromatogram.

TABLE 6

| Time | | |
|---|---|---|
| (minutes) | Identification | Comments |
| 17.4 | Butylated hydroxyl toluene (from | Library match |
| | solvent) | |
| 17.8 | Monoester of succinic anhydride and | Ion = 53, 81, 97, |
| | furfuryl alcohol | 98, 101, 198 |
| 22.1 | Diester of succinic anhydride and | Ions = 53, 81, |
| | furfuryl alcohol | 97, 98, 101, 160 |
| 5.6 | Furfuryl alcohol | Library Match |

The peak at 17.8 had a mass spectrum consistent with the peak detected in Example 2.

The peak at 22.1 had a mass spectrum consistent with the peak detected in Example 2.

The IUPAC name for the product of Example 3 is 4-(furan-2-ylmethoxy)-4-oxobutanoic acid.

### Example 4

A furfuryl-based monoester was synthesized via esterification reaction by reacting furfuryl alcohol and succinic anhydride with the presence of a catalyst. First, 4.91 grams of furfuryl alcohol (0.050 mole) was dissolved in 30 mL of acetone in a 100 mL single-neck round bottom flask 200 along with a magnetic stirring bar 220, as seen in FIG. 2. Then 5.12 grams of succinic anhydride (0.051 mole) was added into the flask 200, and it took about 10 minutes for the white solid to dissolve in the acetone solution. The mixture was a yellow-colored solution. The flask was equipped with a Liebig water-cooled condenser 250. Then 0.60 grams of 4-dimethylaminopyridine (DMAP, 0.005 mole) was added dropwise into the solution. The flask 200 was heated in an oil bath 260 to 65° C. as measured using thermometer 280. The solution was refluxed for 5 hours before the reaction was stopped. The solution was clear and in an amber color. The solvent was removed by a rotary evaporator (Buchi rotavapor R-215, not shown). The final product was an amber colored viscous liquid. The pH value of the final product was pH ˜7, tested by pH test paper. The esterification reaction scheme is shown in FIG. 4.

A sample of Example 4 was diluted with tetrahydrofuran and analyzed by gas chromatography—mass spectrometry [GC/MS]. The analysis conditions are as follows in Table 7:

TABLE 7

| Parameter | Conditions |
|---|---|
| Instrument | Agilent 6890/5973 MSD |
| Column | 30 meter × 0.25 mm RTX-5 with 0.25 micron film |
| | thickness |
| Injection | 2 μL - split ratio 25:1 |
| Carrier | Helium - constant flow @ 1.2 mL/min |
| Program | 50° C. hold 5 minutes, 10° C./minute ramp to 300° C.; |
| | hold 5 minutes |
| Detector | MSD; transfer line 280° C.; source 230° C.; Quad 150° C. |

Peak identification was made based on either a match from the mass spectrum of the peak with commercial database collections (Wiley/NIST) or from similarity with commercial spectra and other data obtained from the analysis. The resulting chromatogram showed 5 main peaks. They are listed in Table 8 in order of their relative peak heights from the total ion chromatogram.

TABLE 8

| Time | | |
|---|---|---|
| (minutes) | Identification | Comments |
| 17.4 | Butylated hydroxyl toluene (from | Library Match |
| | solvent) | |
| 17.8 | Monoester of succinic anhydride and | Ion = 53, 81, 97, |
| | furfuryl alcohol | 98, 101, 198 |
| 22.1 | Diester of succinic anhydride and | Ions = 53, 81, |
| | furfuryl alcohol | 97, 98, 101, 160 |
| 14.8 | 4-dimethylaminopyridine | Library Match |
| 5.6 | Furfuryl alcohol | Library Match |

The peak at 17.8 had a mass spectrum consistent with that seen in Examples 2 and 3.

The peak at 22.1 had a mass spectrum consistent with that seen in Examples 2 and 3.

One other observation noted for Examples 2-4 was that these two products of Examples 3 and 4 were much more viscous that the product of Example 2.

The IUPAC name for the product of Example 4 is 4-(furan-2-ylmethoxy)-4-oxobutanoic acid.

### Example 5

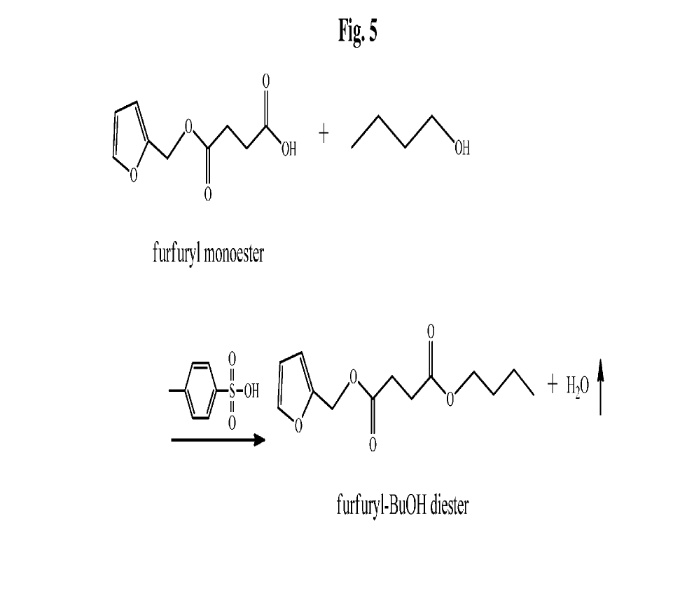

A furfuryl-based diester was synthesized via esterification reaction between the product of Example 4 and 1-butanol in a presence of a catalyst. First, 8.10 grams of furfuryl monoester prepared in Example 4 (0.041 mole) was added into a 250 mL three-neck round bottom flask 100 along with a magnetic stirring bar 120, as seen in FIG. 1. Then 3.22 grams of 1-butanol (0.043 mole) was added and mixed the furfuryl monoester. The mixture was an amber colored viscous liquid. The flask 100 was equipped with a Dean-Stark apparatus 140 connected with a Liebig water-cooled condenser 150. Then the flask 100 was heated in an oil bath 160 to 110° C. as measured using thermometer 180. Then 0.06 grams of p-toluene sulfonic acid (0.5 wt %) was added into the flask 100. The reaction proceeded under dry nitrogen flow through port 190 to help removal of water for 3 hours. The reaction was stopped until the clear liquid collected in Dean-Stark apparatus 140 no longer accumulated. The clear liquid was weighted to be 0.71 grams. The final product was a viscous liquid in amber color. The esterification reaction scheme is shown in FIG. 5.

A sample from Example 5 was diluted with tetrahydrofuran and analyzed by gas chromatography—mass spectrometry [GC/MS]. The analysis conditions are as follows in Table 9:

TABLE 9

| Parameter | Conditions |
|---|---|
| Instrument | Agilent 6890/5973 MSD |
| Column | 30 meter × 0.25 mm RTX-5 with 0.25 micron film |
| | thickness |
| Injection | 2 μL - split ratio 25:1 |
| Carrier | Helium - constant flow @ 1.2 mL/min |
| Program | 50° C. hold 5 minutes, 15° C./minute ramp to 310° C.; |
| | hold 5 minutes |
| Detector | MSD; transfer line 280° C.; source 230° C.; Quad 150° C. |

Peak identification was made based on either a match from the mass spectrum of the peak with commercial database collections (Wiley/NIST) or from similarity with commercial spectra and other data obtained from the analysis. The resulting chromatogram showed 5 main peaks. They are listed in Table 10 in order of their relative peak heights from the total ion chromatogram.

TABLE 10

| Time | | |
|---|---|---|
| (minutes) | Identification | Comments |
| 14.2 | Butylated hydroxyl toluene (from | Library Match |
| | solvent) | |
| 14.4 | Monoester of succinic anhydride and | Ion = 53, 81, 97, |
| | furfuryl alcohol | 98, 101, 198 |
| 17.4 | Diester of succinic anhydride and | Ions = 53, 81, |
| | furfuryl alcohol | 97, 98, 101, 160 |
| 16.0 | Succinic ester of furfuryl alcohol and | Ion = 53, 56, 57, |
| | n-butanol | 81, 97, 98, 101, |
| | | 254 |
| 14.8 | 4-dimethylaminopyridine | Library Match |

The peak at 14.4 had a mass spectrum consistent with that seen in the previous Examples.

The peak at 17.4 had a mass spectrum consistent with that seen in the previous Examples.

The peak at 16 minutes showed a molecular ion of 254, consistent with the desired mixed diester. The peak was Gaussian. The ions detected were also consistent with furfuryl alcohol (53, 81, 97, 98) and succinic acid (100, 101). Ions at 56, and 57, not detected in the other spectra, would be expected for a butyl ester.

The IUPAC name for the product of Example 5 is 1-butyl 4-furan-2-ylmethyl butanedioate

### Examples 6-10

### Quenching Studies

### Example 6

The furfuryl diester prepared in Example 1 was quenched with phenylphosphonic acid as follows. First, 2.00 grams of furfuryl diester (the product of Example 1) was dissolved in 10 mL of acetone in a 30 mL vial. The solution was in an amber color. Then 0.01 grams of phenylphosphonic acid was added and dissolved into the solution. A yellow precipitate formed in around 10 minutes and the solution turned into light yellow. The solution was filtrated via vacuum filtration. The liquid part was analyzed by GC-MS.

### Example 7

### Comparative

The furfuryl diester prepared in Example 1 was quenched with phosphorous acid as follows. First, 2.00 grams of furfuryl diester (the product of Example 1) was dissolved in 10 mL of acetone in a 30 mL vial. The solution was in an amber color. Then 0.01 grams of phosphorous acid was added and dissolved into the solution. A yellow precipitate formed in around 10 minutes and the solution turned into yellow. The solution was filtrated via vacuum filtration. The liquid part was analyzed by GC-MS.

The filtrated liquids of Example 6 and Example 7 were diluted with tetrahydrofuran (THF) and analyzed by GC-MS. The GC-MS chromatogram of filtrated part in Example 6 was found similar to the spectrum of the product prepared in Example 1. The GC-MS chromatogram of filtrated part in Example 7 was found to be primarily starting materials. The significance of these test results was that phosphorus acid was too strong for the furfuryl-based diester.

### Example 8

The quench of catalyst—titanium isopropoxide (Ti(iPro)₄) was further studied as follows. First, 2.03 grams of furfuryl alcohol was added into a 30 mL vial. Then 3 drops of titanium isopropoxide was added. The liquid was initial light yellow and turned into amber color immediately after the catalyst was added. Then 0.30 grams of phenylphosphinic acid was added and mixed by shaking vigorously. The amber liquid turned into clear yellow liquid after 5 minutes. The color stayed the same after 30 minutes in air at room temperature.

The change of color provided information on the interaction between furfuryl alcohol and titanium isopropoxide. The appearance of the amber color indicated the coordination effect of titanium atom with furfuryl alcohol. The disappearance of the amber color suggested the coordination effect between titanium atom and furfuryl alcohol was broken down, and the catalyst was quenched.

### Example 9

The quench of catalyst—titanium isopropoxide (Ti(iPro)₄)—was further studied as follows. First, 2.01 grams of furfuryl alcohol was added into a 30 mL vial. Then 3 drops of titanium isopropoxide was added. The liquid was initial light yellow and turned into amber color immediately after the catalyst was added. Then 0.30 grams of phenylphosphonic acid was added and dissolved into the solution in 3 minutes. The amber colored liquid turned to be clear and then into yellow color in 5 minutes' time. But the color became lighter after 30 minutes, and a precipitate formed as well.

### Example 10

### Comparative

The quench of catalyst—titanium isopropoxide (Ti(iPro)₄) was further studied as follows. First, 2.05 grams of furfuryl alcohol was added into a 30 mL vial. Then 3 drops of titanium isopropoxide was added. The liquid was initial light yellow and turned into amber color immediately after the catalyst was added. Then 0.30 grams of phosphorous acid was added and mixed by shaking vigorously. The solid turned into black in 3 minutes and the amber color got darker. A precipitate formed after 30 minutes, and the color stayed dark and unchanged.

The study in Examples 6-10 of catalyst quenching confirmed that the furfuryl alcohol, when in a complex with titanium isopropoxide (exhibiting a red color), benefits from dissociation from the titanium catalyst upon the introduction of a quenching agent of phenylphosphinic acid or phenylphosphonic acid, but not phosphorus acid. The color of the furfuryl of light yellow returns upon dissociation.

It was also confirmed that diethyl succinate will not form a color complex with either furfuryl alcohol or titanium isopropoxide by (a) mixing diethyl succinate and furfuryl alcohol at a weight ratio of 1:1 resulting in a light yellow color, which comes from furfuryl alcohol and (b) mixing diethyl succinate and titanium isopropoxide (1 wt %) resulting in a light white color due to their miscibility.

### Examples 11-15

### Plasticization of PLA

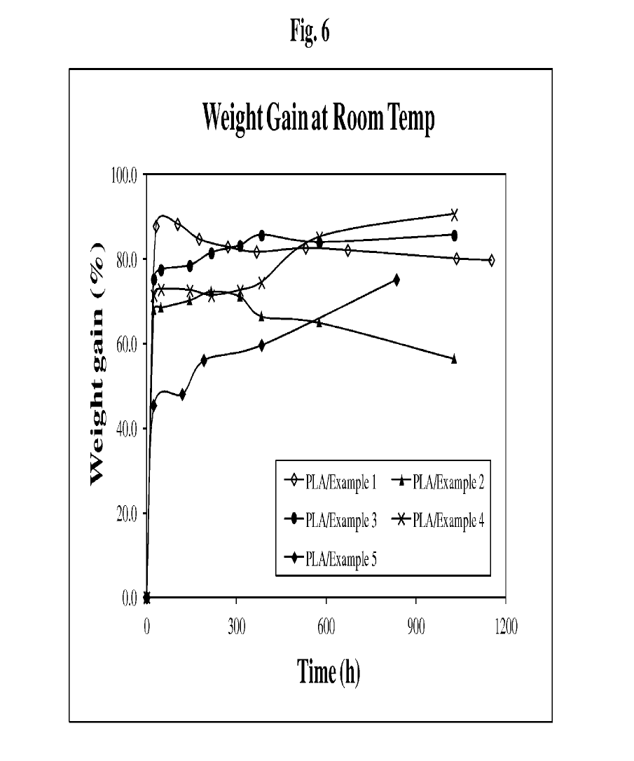

The weight gain of furfuryl diester (product of Example 1), furfuryl monoester (products of Example 2, Example 3, and Example 4) and furfuryl-BuOH diester (product of Example 5) in PLA were studied at room temperature over time. FIG. 6 shows the results.

In each sample, a 5 mm×5 mm square of thin, flat sheet of Ingeo™ 4060D PLA (NatureWorks) was soaked at room temperature in 1 mL of each furfuryl-based ester of Examples 1-5. The weight gains were measured at specific times. The weight gains over time of furfuryl-based esters as plasticizers for PLA at room temperature are shown in FIG. 6.

The weight gain of Example 11 of PLA containing furfuryl diester (product of Example 1) reached 88% in 30 hours, and it remained around 80% over 1600 hours (data point beyond 1600 hours not shown in FIG. 6).

Examples 12-14 of PLA containing furfuryl monoesters (products of Example 2, Example 3, and Example 4, respectively) all reached a weight gain around 70% in 24 hours and achieved a steady state in weight gain over time, except that Example 13 using the furfuryl monoester of Example 3 showed a drop in weight gain after 380 hours.

Example 15 of PLA containing the furfuryl-BuOH diester of Example 5 showed a weight gain of only 45% in 24 hours. Then the weight gain increased gradually and reached 75% after 830 hours.

Based on the overview of these weight gain data, though all five Examples 1-5 could be used as promising plasticizer candidates for PLA, it is indicated from the results that the furfuryl-diester of Example 1 is preferred as a plasticizer for PLA.

The experimental data for Examples 1-5 and 11-15 were also compared with Hansen's 3D solubility parameter calculated by Molecular Modeling Pro 6.33 (provider: ChemSW, Inc.) The Hildebrand solubility parameter for a pure liquid substance is defined as the square root of the cohesive energy density, as described in the following equation:

δ≡[(ΔHᵥ-RT)/Vₘ)]^{1/2}

where ΔHᵥ is the heat of vaporization, and Vₘ the molar volume. RT is the ideal gas pV term, and it is subtracted from the heat of vaporization to obtain an energy of vaporization.

Hansen proposed an extension of the Hildebrand parameter to estimate the relative miscibility of polar and hydrogen bonding systems, using the following equation:

δ²=δ_{d}²+δₚ₊²+δₕ²

where δ is the total solubility parameter, δ_{d}, δₚ, and δₕ are the dispersion, electrostatic, and hydrogen bond components of δ, respectively.

The Hansen's 3D solubility parameters of furfuryl-based esters as plasticizers for PLA are listed in Table 11. The furfuryl diester of Example 1, furfuryl monoester of Examples 2,3,4 (the same product made using different catalysts) and furfuryl-BuOH diester of Example 5 have a total solubility parameter close to each other, suggesting they could be promising candidates as plasticizers for PLA.

As published in an ANTEC paper: Aurus et al., POLYLACTIDES. A NEW ERA OF BIODEGRADABLE POLYMERS FOR PACKAGING APPLICATION. 2005/3240 et seq., FIG. 6 on page 3244, the Hansen 3-D solubility parameter for PLA has been reported to be within a range from about 17 to about 22, with a total solubility parameter at about 19 MPa^{1/2} and a hydrogen bonding component at about 10 MPa^{1/2}.

Surprisingly, for the Formula I of the invention, with n=2 to 22, R=furfuryl, and R′=furfuryl, the Hansen's 3D total solubility parameters calculated using the Molecular Modeling Pro 6.33 software remain within the range from about 17 to about 22.

TABLE 11

| | Hansen's 3-D solubility parameters | | | |
|---|---|---|---|---|
| | (δ² = δ_{d}² + δₚ² + δₕ²) MPa^{1/2} | | | |
| | | | hydrogen | |
| Chemical Name | dispersion | polarity | bonding | total |
| diethyl succinate | 18.36 | 7.64 | 12.45 | 23.46 |
| furfuryl alcohol | 16.80 | 4.66 | 6.92 | 18.75 |
| furfuryl diester of | 18.98 | 5.35 | 11.23 | 22.69 |
| Example 1 | | | | |
| furfuryl monoester | 18.03 | 5.05 | 8.75 | 20.67 |
| of Examples 2-4 | | | | |
| furfuryl-BuOH | 18.04 | 4.38 | 8.55 | 20.44 |
| diester of Example 5 | | | | |
| Formula I with n = 2, | 18.98 | 5.34 | 11.23 | 22.69 |
| R = furfuryl, | | | | |
| R′ = furfuryl (same | | | | |
| as Example 1) | | | | |
| Formula I with n = 6, | 16.16 | 4.89 | 9.95 | 19.60 |
| R = furfuryl, | | | | |
| R′ = furfuryl | | | | |
| Formula I with n = 8, | 16.27 | 4.47 | 9.95 | 15.59 |
| R = furfuryl, | | | | |
| R′ = furfuryl | | | | |
| Formula I with | 15.01 | 4.82 | 11.38 | 19.44 |
| n = 10, R = furfuryl, | | | | |
| R′ = furfuryl | | | | |
| Formula I with | 17.98 | 4.51 | 11.69 | 21.91 |
| n = 18 R = furfuryl, | | | | |
| R′ = furfuryl | | | | |
| Formula I with | 18.77 | 4.20 | 11.68 | 22.50 |
| n = 22, R = furfuryl, | | | | |
| R′ = furfuryl | | | | |

The total Hansen's 3D solubility parameter for each of Examples 1-5 is close, but not too close, to the total solubility parameter for PLA at its peak as reported by Aurus et al. Too close to the peak can result in undesirable solubilization of the polymer merely meant to be plasticized.

This determination seen in Table 11 confirms the appropriateness of these monoesters and diesters as plasticizers for PLA. As reported in Carraher et al., Introduction to Polymer Chemistry 2009, page 55, “Through experience, it is found that the solubility parameter difference between the plasticizer and the polymer should be less than 1.8H.” 1.8H is correlated to be 3.7 MPa^{1/2}. One of the monoesters and diesters of the present invention can satisfy this Carraher proposition for a polymer having a total solubility parameter of between about 17 and about 26 MPa¹″².

The calculations for n>6, beyond the range of actual experimental results, is not merely theoretical. There are bio-derived diacids up to n=14 currently commercially available from Cathay Industrial Biotech Ltd. of Shanghai, China, and it is expected that the bio-derived diacids will become available in the future.

### Examples 16-21

### Extrusion of Diesters with PLA

Compounds of PLA/furfuryl-diester plasticizers of Example 1 were made on Prism 16 mm extruder operating at 150° C. in all zones and die, 200 rpm, a die pressure of 2 bar, and a feeder rate of 7%, under vacuum. The formulations are listed in Table 12 below.

Example 16, a comparative example, was neat PLA to serve as a control. Example 17, was a combination of PLA and furfuryl diester of Example 1 at a 3:1 weight ratio. Alternatively expressed, the furfuryl alcohol was 25 weight percent of the compound or 33 parts per hundred of PLA polymer. Examples 18-21 used the same proportions of PLA and furfuryl diester.

Because the diester used was Example 1, which employed the titanium isopropoxide catalyst, the remaining Examples 18-21 studied the timing of quenching of the complex of furfuryl alcohol and titanium catalyst studied in Examples 6-10 above. For Examples 18 and 19, the two types quenching agents were mixed with PLA pellets for each batch during extrusion. For Examples 20 and 21, the quenching agents were added into the furfuryl-based diester plasticizer before extrusion began. These pre-quenched plasticizers were allowed to sit overnight. Precipitates formed in the pre-quenched plasticizer in Example 21 using phenylphosphonic acid, and the precipitates were filtered off before extrusion began.

One day before the experiments, the PLA was stored in dry conditions to minimize effects of moisture upon the extrusion.

The liquid plasticizer for all Examples 17-21 was added downstream using a liquid injection feeder operating at 3.5 rpm. The vacuum level was about 19-20 kPa for all Examples 16-21.

TABLE 12
Extrusions of PLA and Furfuryl Plasticizer

| Example | 16* | 17 | 18 | 19 | 20 | 21 |
|---|---|---|---|---|---|---|
| PLA 4060 D (dry) | 100 | 75 | 74.3 | 74.3 | 75 | 75 |
| Furan-diester plasticizer | | 25 | 25 | 25 | | |
| Quench agent1- | | | 0.7 | | | |
| phenylphosphinic acid | | | | | | |
| Quench agent2- | | | | 0.7 | | |
| phenylphosphonic acid | | | | | | |
| Pre-quenched furan- | | | | | 25 | |
| diester with | | | | | | |
| phenylphosphinic acid | | | | | | |
| Pre-quenched furan- | | | | | | 25 |
| diester with | | | | | | |
| phenylphosphonic acid | | | | | | |
| Total | 100 | 100 | 100 | 100 | 100 | 100 |
| Tg (° C.) | 59.8 | 5.8 | 4.1 | 10.4 | 8.9 | 14.4 |

*Comparative

Strands of Example 16 were clear and rigid. Strands of Examples 17-19 were flexible and transparent but amber-colored and turned gradually hazy after one week. Compounds of Examples 20 and 21 were flexible strands but a little hazy upon emerging from the extruder.

None of Examples 17-21 showed any indication of blooming of plasticizer to the surface of the strands. The compounds of Examples 16-21 were analyzed by DSC to obtain glass transition temperatures, as seen in Table 12 above.

The samples of Examples 16-21 were analyzed by differential scanning calorimetry (DSC) using a TA Instruments model DSC Q50. Each specimen was exposed to a heat-cool-heat cycle in the analysis. The first heating scan typically contains thermal events reflecting thermal/processing history. The controlled cooling provided an established thermal history and allowed determinations of the transitions based on material properties in the second heating scan. The temperature range of each segment was from 40° C. to 210° C. at heating/cooling rates of 10° C./minute. A nitrogen gas purge of 50 ml/minute was used. The glass transition temperature (Tg) of each sample was determined using the half-height from the data recorded in the second heating segment of the analysis.

Using Example 16 as a control, the Tg of PLA is too high to be flexible at room temperature. By comparison, the Tg results for Examples 17-21 all demonstrate good flexibility and plasticization of PLA by any of the means of extruding the furfuryl-based diester of Example 1 with PLA. The unquenched Example 17 performed as well as the quenched Examples 18-21 by a variety of quenching means and quenching agents.

The novel furfuryl-based monoesters and furfuryl-based diesters of the present invention are good candidates for a variety of polymers having a total Hansen's 3D solubility parameter from about 17 to about 26. Without undue experimentation, a person having ordinary skill in the art can select a furfuryl-based ester, a quenching agent (if using an organometallic catalyst), and variety of means of melt mixing to prepare plasticized polymer compounds.

Because the novel furfuryl-based monoesters and furfuryl-based diesters of the present invention can be synthesized using bio-derived starting materials, these monoesters and diesters qualify as new sustainable plasticizers for use in a variety of industries.

The invention is not limited to the above embodiments. The claims follow.

What is claimed is:

1. A composition of matter, comprising: 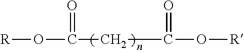
wherein n=2 to 22, inclusive; R=furfuryl; and R′=hydrogen or an alkyl having from 2 to 10 carbon atoms.

2. The composition of matter of claim 1, wherein R′ is hydrogen, and wherein the composition of matter is a monoester.

3. The composition of matter of claim 1, wherein R′ is an alkyl having from 2 to 10 carbon atoms, and wherein the composition of matter is a diester.

4. The composition of matter of claim 1, wherein the composition of matter selected from the group consisting of 4-(furan-2-ylmethoxy)-4-oxobutanoic acid and 1-butyl 4-furan-2-ylmethyl butanedioate.

5. The composition of matter of claim 4, wherein the composition of matter is 4-(furan-2-ylmethoxy)-4-oxobutanoic acid.

6. The composition of matter of claim 4, wherein the composition of matter is 1-butyl 4-furan-2-ylmethyl butanedioate.

7. A composition of matter comprising: 1,4-bis(furan-2-ylmethyl) butanedioate synthesized by an organometallic-catalyzed transesterification reaction of furfuryl alcohol and diethyl succinate in the presence of titanium isopropoxide.

8. The composition of matter of claim 5, wherein the 4-(furan-2-ylmethoxy)-4-oxobutanoic acid is synthesized by a base-catalyzed ring-opening esterification reaction of furfuryl alcohol and succinic anhydride in the presence of a catalyst selected from the group consisting of pyridine, triethylamine, and dimethylaminopyridine.

9. The composition of matter of claim 6, wherein the 1-butyl 4-furan-2-ylmethyl butanedioate is synthesized by an acid-catalyzed direct esterification reaction of a furfuryl monoester and 1-butanol in the presence of p-toluene sulfonic acid.

10. The composition of matter of claim 7, wherein the titanium isopropoxide is quenched by either phenylphosphinic acid or phenyiospsphonic acid.

11. A thermoplastic compound, comprising a polymer plasticized by a composition of matter comprising: 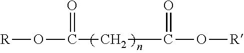
wherein n=2 to 22, inclusive; R=furfuryl; and R′=hydrogen, furfuryl, or an alkyl having from 1 to 10 carbon atoms.

12. The compound of claim 11, wherein the polymer is polylactic acid.

13. The compound of claim 11, wherein the polymer is polyvinyl chloride.

14. The compound of claim 11, wherein the polymer has a Hansen's 3D Solubility Parameter ranging from 17 to 26.

15. The compound of claim 11, wherein the plasticizer is present in the polymer from about 10 to about 100 parts per hundred of polymer.

16. The compound of claim 15, wherein the compound further comprises additives selected from the group consisting of adhesion promoters; biocides; anti-fogging agents; anti-static agents; bonding, blowing and foaming agents; crosslinking agents; dispersants; fillers and extenders; fire and flame retardants; smoke suppressants; impact modifiers; initiators; lubricants; micas; colorants; additional plasticizers; processing aids; release agents; silanes; titanates; zirconates; slip agents; anti-blocking agents; stabilizers; stearates; ultraviolet light absorbers; viscosity regulators; waxes; and combinations of them.

17. A plastic article formed from the compound of claim 11.

18. A reaction product of the composition of matter of claim 1 at R with a dienophile.

19. A reaction product of the composition of matter of claim 1 at R′ with a chemical,
wherein if R′ is furfuryl, the chemical is a dienophile;
wherein if R′ is hydrogen, the chemical has a functional group selected from the group consisting of an alcohol, an oxirane, an amine, a metallic ion, a hydroxide, a silane and a silanol; and
wherein if R′ is an alkyl, the chemical is an ester reactive via transesterification in the presence of a catalyst.

20. The compound of claim 14, wherein the plasticizer is present in the polymer from about 10 to about 100 parts per hundred of polymer.
